(19) United States
(12) Patent Application Publication
(10) Pub. No.: US 2022/0313415 A1
(43) Pub. Date: Oct. 6, 2022

(54) DEVICE SYSTEM AND METHOD FOR BLUNT TISSUE DISSECTION AND STRESS URINARY INCONTINENCE TREATMENT

(71) Applicant: OORO MEDICAL LTD, Rosh Ha'ayin (IL)

(72) Inventors: Eyal SANDACH, Yahud (IL); Boaz HARARI, Ganey Tikva (IL)

(73) Assignee: OORO MEDICAL LTD, Rosh Ha'ayin (IL)

(21) Appl. No.: 17/596,970

(22) PCT Filed: Jun. 23, 2020
(86) PCT No.: PCT/IL2020/050696

§ 371 Date: Dec. 22, 2021

Related U.S. Application Data
(60) Provisional application No. 62/865,351, filed on Jun. 24, 2019.

(51) Int. Cl. A61F 2/00; A61B 17/32

(52) U.S. Cl. CPC A61F 2/0045; A61B 17/320016; A61B 2017/320044

(57) ABSTRACT

A surgical device for blunt tissue dissection is provided as are a guide and delivery plunger that can be used along with the surgical device to deliver an implant in a tissue repair procedure.

### BACKGROUND

The present invention relates to a device for blunt tissue dissection and a system using same for delivery of an implant to a target tissue. Embodiments of the present invention relate to a device and system that can be used in treatment of stress urinary incontinence.

Numerous surgical procedures require establishment of a tissue path to target tissues to effect repair. For example, sub-mid urethral sling placement and fixation for treatment of incontinence requires access to the obturator area through a para-vesical bilateral dissection in order to anchor an implant to a solid pelvic structure.

Although such a procedure is commonly practiced, vaginal access to the obturator area is challenging and can be risky since organs and tissues surrounding the access path can easily be injured during dissection.

There is thus a widely recognized need for, and it would be highly advantageous to have, an approach devoid of the above limitations.

### SUMMARY

According to one aspect of the present invention there is provided a surgical device for positioning of an implant comprising an elongated device body having a tissue dissector at a distal end and a handle for actuating the tissue dissector at a proximal end, the elongated device body being curved along its length.

According to another aspect of the present invention there is provided a guide comprising a handle attached to a curved guide tube configured for containing said elongated device body.

According to another aspect of the present invention there is provided system for positioning of an implant comprising a flexible elongated device body having a tissue dissector at a distal end and a handle for actuating said tissue dissector at a proximal end; and a guide including a guide handle attached to a curved guide tube configured for containing said elongated device body.

According to another aspect of the present invention there is provided a method of positioning an implant in a target tissue comprising delivering an implant to a target tissue using a blunt tissue dissector positioned through a slotted lumen of a guide having a curved guide tube; and freeing the implant from the guide through a slot of the slotted lumen of the guide tube.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates one embodiment of a system for blunt dissection and implant delivery constructed in accordance with the teachings of the present invention.

FIG. 2 illustrates one embodiment of the blunt dissector of the present invention.

FIGS. 3A-B illustrate the blunt dissecting head in an open (FIG. 3A and closed (FIG. 3B) positions.

FIGS. 4A-B and 5 illustrate another embodiment of a system for blunt dissection and implant delivery constructed in accordance with the teachings of the present invention showing the blunt dissector and guide separately (FIGS. 4A-B respectively) and engaged (FIG. 5).

FIGS. 6-7 illustrate one embodiment of a guide constructed in accordance with the teachings of the present invention.

FIG. 8 is an exploded view of one embodiment of the guide and blunt dissector of the present invention.

FIG. 9 is a side cutaway view of one embodiment of the blunt dissector of the present invention.

FIGS. 10-11 illustrate delivery of an implant through the guide using a plunger.

### DETAILED DESCRIPTION

The present invention is of a system which can be used to dissect a tissue path to an anatomical landmark and deliver and implant thereto. Specifically, the present invention can be used for treatment of stress urinary incontinence.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Tissue reconstruction is a key for stress urinary incontinence repair. Reconstruction can be effected by anchoring a supportive implant (suture, mesh) to the obturator complex (fascia, muscles etc.) positioned at the antero-lateral aspect of the pelvis. Access to the obturator is via a surgical cut at the mid-line of the anterior vaginal wall and lateral dissection under the vaginal wall towards the pelvic side wall and the obturator complex. Such a surgical approach can be dangerous and requires proper training and skill.

Blunt dissection under the vaginal wall can provide access to the obturator with minimal tissue trauma and danger. Although devices that can be used to blunt dissect tissue are common (e.g. blunt dissection forceps), such devices are designed for basic blunt dissection operability and as such, their functionality and adaptability are limited.

In order to overcome the limitations of prior art blunt dissectors, the present inventors have devised a surgical device that can provide a solution for surgical approaches that require blunt dissection of tissue along a curved path, while minimizing tissue trauma. As is further described hereinunder, the blunt tissue dissector of the present invention can be curved or used with a curved guide in a system that can be used to establish a curved dissection path and deliver an implant such as a suture or mesh sling.

Thus, according to one aspect of the present invention, there is provided a surgical device which can be used for blunt dissection of tissues, such as, for example, muscle, fat, connective tissue and the like of a treated subject such as a human.

The surgical device (also referred to herein as a “blunt dissector”) includes an elongated device body having a tissue dissector at a distal end and a handle for actuating the tissue dissector at a proximal end.

The elongated device body (also referred to herein as “shaft”) can be 6-12 cm in length and 3-5 mm in diameter.

The shaft can be straight or curved along its length. The radius of curvature of the curved shaft can be 8-20 cm. The elongated device body can be fabricated as a tube from a polymer or alloy using well known approaches.

The blunt dissecting head can include a pair of movable jaws that can be actuated between closed and open positions using the handle. The handle can be configured to be operated via a hand grasp-release or a push-pull-type interface.

As is mentioned hereinabove, the present dissector can be used to establish a curved dissection path. A curved dissection path can be formed using a curved (shaft) dissector or a straight (shaft) dissector having a flexible shaft that can assume a curved configuration when positioned within a curved guide.

The guide can include a guide tube with a radius of curvature of 8-20 cm and a slotted lumen that can accommodate the shaft of the blunt dissector device.

The guide serves two purposes, guiding of the blunt tissue dissector to the vaginal wall and following dissection of a path, delivery of an implant to the anatomical landmark (e.g. obturator).

As is further described hereinbelow, the slotted lumen facilitates release of a suture or sling delivered through the lumen of the guide tube using a plunger/pushrod.

Although a curved guide is presently preferred, it will be appreciated that a system including a straight blunt dissector positioned through a straight guide can also be used for blunt tissue dissection and implant delivery.

Although the present blunt tissue dissector (straight or curved shaft) can be used as a standalone device to provide a dissection path to the anatomical landmark of choice, it is preferably used as a part of a system that includes the guide. As is further described hereinunder, such a system can be used to safely establish an accurate dissection path while enabling easy and rapid implant delivery.

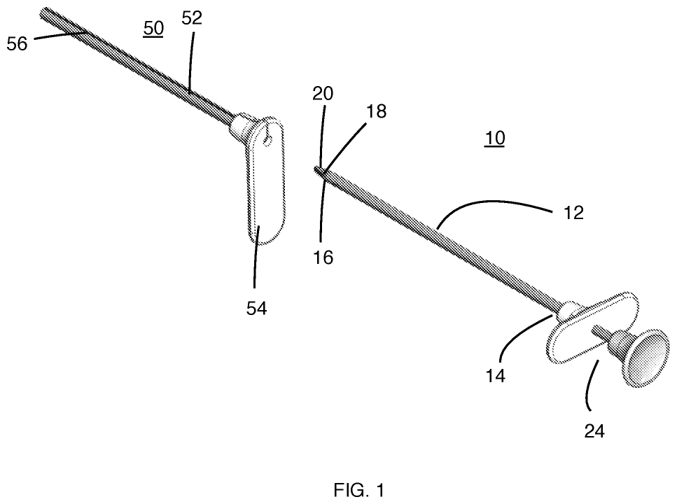

Referring now to the drawings, FIGS. 1-11 illustrate embodiments of the present system, surgical device and guide which are referred to herein as surgical device 10, guide 50 and system 100.

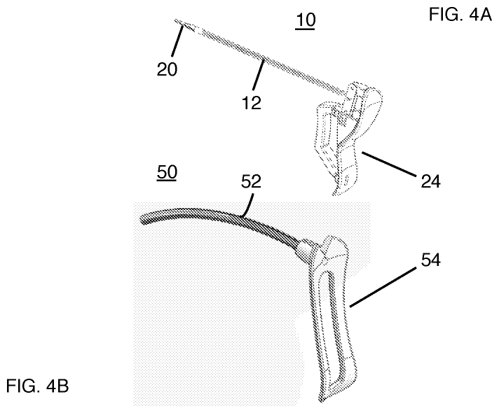

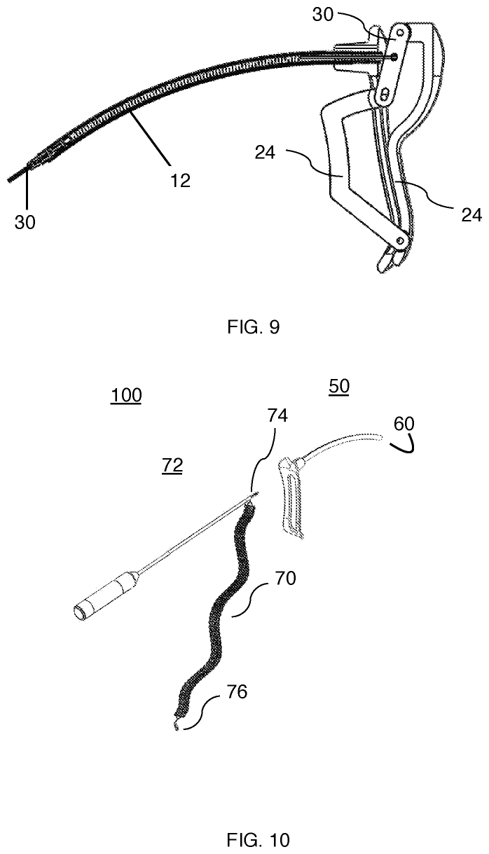

Device 10 includes an elongated device body 12 (also referred to herein as “shaft 12”) having a proximal end 14 and a distal end 16. Shaft 12 can be linear as is shown in FIG. 1, curved as is shown in FIG. 5 or flexible (FIG. 4A) and capable of assuming a curved configuration as is shown in FIGS. 4A and 9.

The curved configuration of shaft 12 can be fabricated from a spiral/helical wire so as to enable elastic bending/flexing of shaft 12 when delivered through a curved configuration of guide 50 (FIG. 4B).

Shaft 12 carries a blunt dissector head 18 at distal end 16. Blunt dissector head 18 includes tissue biasing element(s) that are preferably configured as one or more pair(s) of movable jaws 20 (one pair shown) which pivot in a scissor-like action around pin 22. Jaws 20 are configured for blunt tissue dissection by outwardly biasing tissue and as such are flat and blunt. The biasing (outward) force applied by jaws 20 can be in the range of 100-1000 grams. Jaws 20 can be made out of any metal or alloy (e.g. stainless steel, titanium) or any other rigid material.

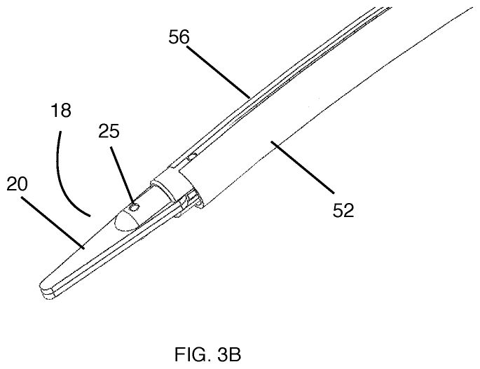

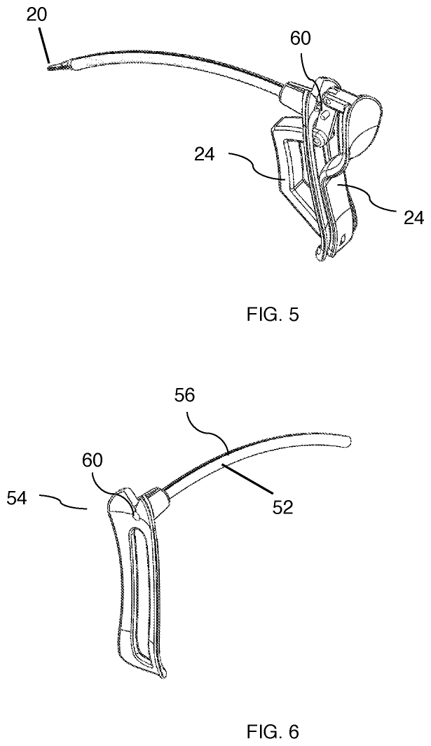

Jaws 20 are actuatable between open (FIG. 3A) and closed (FIG. 3B) positions (and anywhere in between) via a manual or motorized actuating mechanism that is actuated via handle 24 (further described hereinbelow with respect to FIGS. 6 and 9).

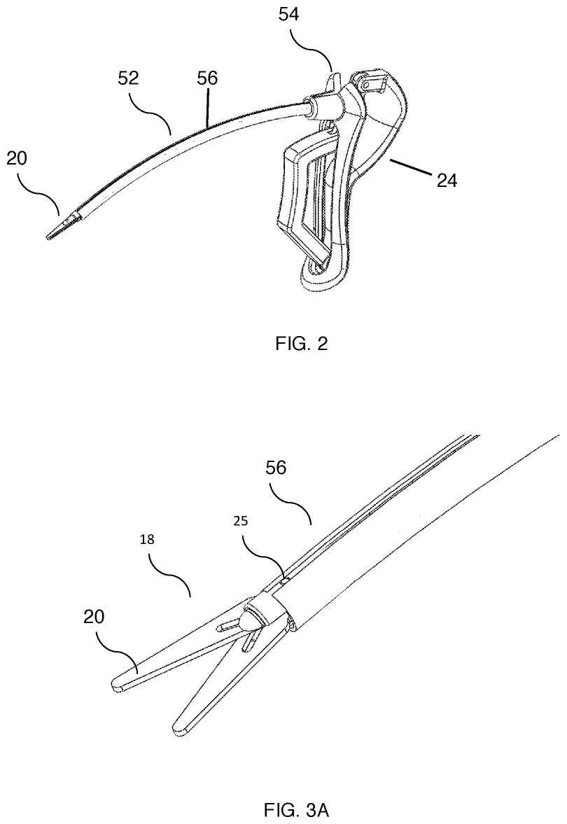

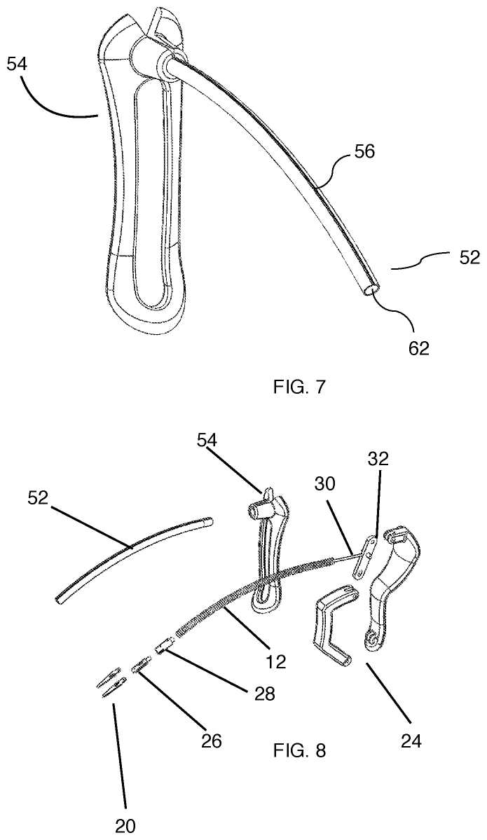

Handle 24 can have a push-release (FIG. 1) or grasp-release (FIG. 2) configuration. Actuation of blunt dissector head 18 can be manual or motorized through rod(s) or wire(s) positioned through shaft 12. Further description of the operation and internal mechanism of device 10 is provided with respect to FIGS. 8 and 9 below.

As is shown in FIGS. 8 and 9, jaws 20 are hinged at distal jaw base 26 by pin 25 (FIGS. 3A-B). Base 26 is connected to shaft 12 that can be rigid or flexible to allow its passage through guide tube 52. Jaw slider 28 is actuated via push\pull rod 30 that moves jaws 22 between open and closed positions. Rod 30 is actuated by lever mechanism 32.

As is mentioned hereinabove, device 10 can be used along with guide 50 (collectively forming system 100) in repair of tissue (e.g. repair pelvic floor prolapse). Embodiments of system 100 are shown in FIGS. 1, 2, 4 and 5.

Guide 50 is shown alone in FIGS. 1, 6 and 7. Guide 50 includes a guide tube 52 attached to a handle 54. Guide tube is preferably curved and includes a slotted lumen 56 running the length of guide tube 52. Slot 58 is dimensioned so as to enable release of a suture/sling delivered through lumen 56 without having to remove guide 50 from the tissue (dissection path). The internal diameter of lumen 56 can be about 3-8 mm in diameter to accommodate shaft 12 of device 10 (as is shown in FIGS. 2 and 5). Lumen 56 includes a proximal opening 60 and a distal opening 62 in order to enable shaft 12 of device 10 to be positioned through guide tube 52. Slot 58 can be about 0.4-0.7 mm in width and runs the length of guide tube 52.

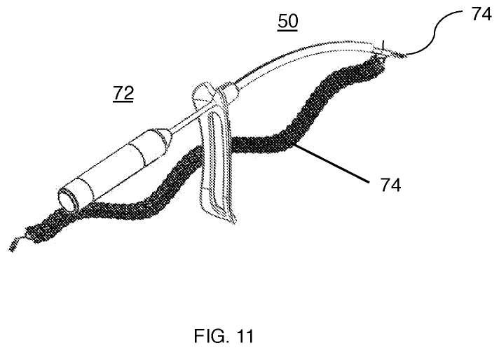

Guide 50 is used along with device 10 to dissect a path to an anatomical landmark (e.g. obturator). Once the path is established using device 10 (positioned in guide 50), device 10 is removed while maintaining guide 50 in position in the dissection path with distal opening 62 positioned at or near the target tissue. As is shown in FIGS. 10-11, an implant 70 (mesh sling shown) attached to a plunger or pushrod 72 having a tissue piercing end 74 can be inserted into lumen 56 through proximal opening 60. Plunger or pushrod 72 can then be used to anchor implant 70 into the target tissue following which, the plunger or pushrod 72 can be pulled out of guide 50. The opposite end of implant 70 can include a hook/loop 76 for anchoring it to the same or different tissue structure using plunger 72 and optionally guide 50. Guide 50 can then be removed from the body (tissue dissection path) while maintaining implant 70 in position and releasing it from guide 50 through slot 58.

System 100 of the present invention can be used to blunt dissect any tissue of any organ or body region and deliver any implant to target tissues. For example, system 100 of the present invention can be used in surgical procedures such as anterior prolapse dissection, male anti-incontinence surgery, orthopedic reconstruction of torn ligaments, cosmetic surgery and the like.

One presently preferred use of system 100 of the present invention is in pelvic floor prolapse repair procedures. Specifically, system 100 can be used to blunt dissect a tissue path to the obturator complex and guide an implant thereto.

System 100 can be used to create a dissection path to the obturator and deliver an implant as follows.

Briefly, a saline mixture which optionally includes a local anesthetic [e.g., xylocaine 1%+/−adrenalin 0.2 ml (1000 units/ml)] is infiltrated locally via 20 ml injections 2-4 mm under the anterior vaginal wall at the sub mid urethral region. An incision of 1 cm is made at the site of infiltration. Dissection using device 10 starts from midline, below the vaginal wall and underlying fascia, through the areolar tissue toward the pelvic side wall (sub-obturator region) for a distance of about 5 cm (arrow 1, FIG. 11).

A suture, anchor or mesh can then be guided along channel 40 (as described above) and anchored in the obturator complex using anchors sutures or the like. Fixation of the mesh to the soft tissue can be achieved by suturing using a suture and needle or a suturing device, by anchoring with a harpoon, by tuckers or by any other combinations thereof. The suture or anchors can be long standing or absorbable. The mesh can be made of any synthetic, biologic or organic material, long standing or absorbable, plain, knighted or woven, placed for reinforcing the native tissue contributing to the herniation/prolapse/relaxation of the pelvic floor.

System 100 can also be used to position a sub mid-urethral support device such as a sling, mesh, grid and the like formed from a synthetic polymer or autologous or non-autologous biological tissue; such a device can be permanent, absorbable or partially absorbable.

The free end of the anchored suture (described above) can be attached to the sub mid-urethral support device or to the para-urethral tissue. Such attachment can be via suturing, gluing, clipping, tying, knotting, buttoning or by any other method.

When choosing not to use any sub urethral support device, the procedure is carried out exactly as with the supporting device until both the fixation sutures are anchored laterally to the obturator tissues elected for this purpose, passing under the vaginal wall up to the sub mid-urethral vaginal wall small incision, then carried out to the vaginal cavity. The free ends of the sutures are then fixed to the para urethral tissue on both sides via, for example, suturing, buttoning, gluing, tying, clipping, or the like.

As used herein the term “about” refers to ±10%.

Additional objects, advantages, and novel features of the present invention will become apparent to one ordinarily skilled in the art upon examination of the following examples, which are not intended to be limiting.

### Examples

Reference is now made to the following example, which together with the above descriptions, illustrate the invention in a non-limiting fashion.

### Patient Procedure

A procedure was performed using the present surgical device. A 4 mm incision was made through the anterior vaginal wall under the mid urethra under local anesthesia. A mini-dissection was performed between the vaginal wall and bladder using the present dissector and guide, starting at the incision site and ending at the inferior ramus of the pubic bone. The dissector was removed and an anchor was delivered through the guide channel to the obturator membrane. The procedure was repeated on the contra-lateral side, the mesh implant was adjusted and the incision was closed using an absorbable suture.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A surgical device for positioning of an implant comprising an elongated device body having a tissue dissector at a distal end and a handle for actuating said tissue dissector at a proximal end, said elongated device body being curved along its length.

2. The device of claim 1, wherein a radius of curvature of said elongated device body is 8-20 cm.

3. The device of claim 1, wherein said tissue dissector is a blunt tissue dissector having a pair of movable jaws.

4. The device of claim 1, wherein said handle includes a push-pull interface.

5. A system comprising the device of claim 1 and a guide including a guide handle attached to a guide tube configured for containing said elongated device body.

6. The system of claim 5, wherein said guide tube includes a slotted lumen.

7. The system of claim 5, wherein said guide tube is curved along its length.

8. The system of claim 7, wherein said elongated device body is linear and assumes a curved configuration when constrained within said guide tube.

9. The system of claim 1, further comprising a pushrod positionable within said lumen.

10. A system for positioning of an implant comprising:
(a) an elongated device body having a tissue dissector at a distal end and a handle for actuating said tissue dissector at a proximal end; and
(b) a guide including a guide handle attached to a guide tube configured for containing said elongated device body.

11. The system of claim 10, wherein said guide tube includes a slotted lumen.

12. The system of claim 10, wherein said guide tube is curved along its length.

13. The system of claim 12, wherein said elongated device body is linear and assumes a curved configuration when constrained within said guide tube.

14. A method of positioning an implant in a target tissue comprising:
(a) using the system of claim 11 to blunt dissect a path to the target tissue.
(b) delivering the implant to the target tissue through a lumen of said guide; and
(c) freeing the implant from said guide through a slot of said slotted lumen.

15. The method of claim 14, wherein (b) is effected by a plunger positioned in said lumen.

16. The method of claim 15, wherein said plunger includes a tissue piercing distal end for embedding the implant within the target tissue.

17. The method of claim 14, wherein the target tissue is the obturator and the implant is a suture or a mesh sling.
